(19) United States
(12) Patent Application Publication
(10) Pub. No.: US 2024/0331824 A1
(43) Pub. Date: Oct. 3, 2024

(54) COGNITIVE CARDIAC AUSCULTATION BASED ON MULTIMODALITY AND CONTRASTIVE LEARNING

(71) Applicant: INTERNATIONAL BUSINESS MACHINES CORPORATION, ARMONK, NY (US)

(72) Inventors: Si Tong Zhao, Beijing (CN); Li Juan Gao, Xi'an (CN); Yuan Yuan Ding, Shanghai (CN); Tong Liu, Xi'an (CN)

(21) Appl. No.: 18/190,247

(22) Filed: Mar. 27, 2023

(51) Int. Cl. G16H 20/00; G16H 40/67; G16H 50/20

(52) U.S. Cl. CPC G16H 20/00; G16H 40/67; G16H 50/20

(57) ABSTRACT

A method, computer program product, and computer system are provided for predicting treatment options based on cardiac auscultation data. Text data and audio data corresponding to cardiac auscultation associated with a patient is received. The text data and the audio data are encoded as respective text vectors and audio vectors. A distance between the text vectors and the audio vectors is calculated. Diagnosis results are determined by a machine learning model based on the calculated distance between the text vectors and the audio vectors.

### FIELD

This disclosure relates generally to the field of machine learning, and more particularly to medical diagnostics through machine learning.

### BACKGROUND

In the medical field, heart sound auscultation is a very common diagnostic method through which cardiovascular and other diseases may be identified by listening to heart sounds. The traditional method is that doctors make a diagnosis through a stethoscope during diagnosis and treatment. With the development of technology, some intelligent stethoscope device gradually appeared in recent years, that is, the stethoscope analyzes the heart sound through the method of machine learning or deep learning after collecting the heart sound, and directly provides the result of the intelligent diagnosis for the reference of the doctor.

### SUMMARY

Embodiments relate to a method, system, and computer program product for predicting treatment options based on cardiac auscultation data. According to one aspect, a method for predicting treatment options based on cardiac auscultation data is provided. The method may include receiving text data and audio data corresponding to cardiac auscultation associated with a patient. The text data and the audio data are encoded as respective text vectors and audio vectors. A distance between the text vectors and the audio vectors is calculated. Diagnosis results are determined by a machine learning model based on the calculated distance between the text vectors and the audio vectors.

According to another aspect, a computer system for predicting treatment options based on cardiac auscultation data is provided. The computer system may include one or more processors, one or more computer-readable memories, one or more computer-readable tangible storage devices, and program instructions stored on at least one of the one or more storage devices for execution by at least one of the one or more processors via at least one of the one or more memories, whereby the computer system is capable of performing a method. The method may include receiving text data and audio data corresponding to cardiac auscultation associated with a patient. The text data and the audio data are encoded as respective text vectors and audio vectors. A distance between the text vectors and the audio vectors is calculated. Diagnosis results are determined by a machine learning model based on the calculated distance between the text vectors and the audio vectors.

According to yet another aspect, a computer program product for predicting treatment options based on cardiac auscultation data is provided. The computer program product may include one or more computer-readable storage devices and program instructions stored on at least one of the one or more tangible storage devices, the program instructions executable by a processor. The program instructions are executable by a processor for performing a method that may accordingly include receiving text data and audio data corresponding to cardiac auscultation associated with a patient. The text data and the audio data are encoded as respective text vectors and audio vectors. A distance between the text vectors and the audio vectors is calculated. Diagnosis results are determined by a machine learning model based on the calculated distance between the text vectors and the audio vectors.

### BRIEF DESCRIPTION OF THE DRAWINGS

These and other objects, features and advantages will become apparent from the following detailed description of illustrative embodiments, which is to be read in connection with the accompanying drawings. The various features of the drawings are not to scale as the illustrations are for clarity in facilitating the understanding of one skilled in the art in conjunction with the detailed description. In the drawings:

FIG. 1 illustrates a networked computer environment according to at least one embodiment;

FIG. 2 illustrates a networked computer environment according to at least one embodiment

FIG. 3 is a block diagram of a system for predicting treatment options based on cardiac auscultation data, according to at least one embodiment; and

FIG. 4 is an operational flowchart illustrating the steps carried out by a program that predicts treatment options based on cardiac auscultation data, according to at least one embodiment.

### DETAILED DESCRIPTION

Detailed embodiments of the claimed structures and methods are disclosed herein; however, it can be understood that the disclosed embodiments are merely illustrative of the claimed structures and methods that may be embodied in various forms. Those structures and methods may, however, be embodied in many different forms and should not be construed as limited to the exemplary embodiments set forth herein. Rather, these exemplary embodiments are provided so that this disclosure will be thorough and complete and will fully convey the scope to those skilled in the art. In the description, details of well-known features and techniques may be omitted to avoid unnecessarily obscuring the presented embodiments.

Embodiments relate generally to the field of machine learning, and more particularly to medical diagnostics through machine learning. The following described exemplary embodiments provide a system, method, and computer program product to, among other things, predict treatment options based on cardiac auscultation data. Therefore, some embodiments have the capacity to improve the field of computing by allowing for diagnosis of heart defects and other conditions without fine-grained annotation data.

As previously described, in the medical field, heart sound auscultation is a very common diagnostic method through which cardiovascular and other diseases may be identified by listening to heart sounds. The traditional method is that doctors make a diagnosis through a stethoscope during diagnosis and treatment. With the development of technology, some intelligent stethoscope device gradually appeared in recent years, that is, the stethoscope analyzes the heart sound through the method of machine learning or deep learning after collecting the heart sound, and directly provides the result of the intelligent diagnosis for the reference of the doctor.

At present, most of the intelligent stethoscope background algorithms are based on the signal analysis method and the deep learning model method of acoustic classification. However, because the heart sound signal is weak and easily disturbed by the external environment, the accuracy is still low; and the deep learning model usually requires a large amount of fine-grained labeling data, labeling of sound signals is time-consuming and requires labeling by professionals with a deep understanding of cardiac disease. It may be advantageous, therefore, to use an unsupervised deep learning method based on contrastive learning and multimodality, which can diagnose heart sound signals without fine-grained annotation. It provides a diagnosis and treatment plan for inexperienced doctors, and patients can also obtain the diagnosis results by themselves with the help of this product. Only the corresponding heart sound audio data and medical record text data are needed, and there is no need to mark what happened in the time period of the audio data. This method can save manpower, and due to the unsupervised nature of the model, there may be ample training data, so long as there is enough time to collect data.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

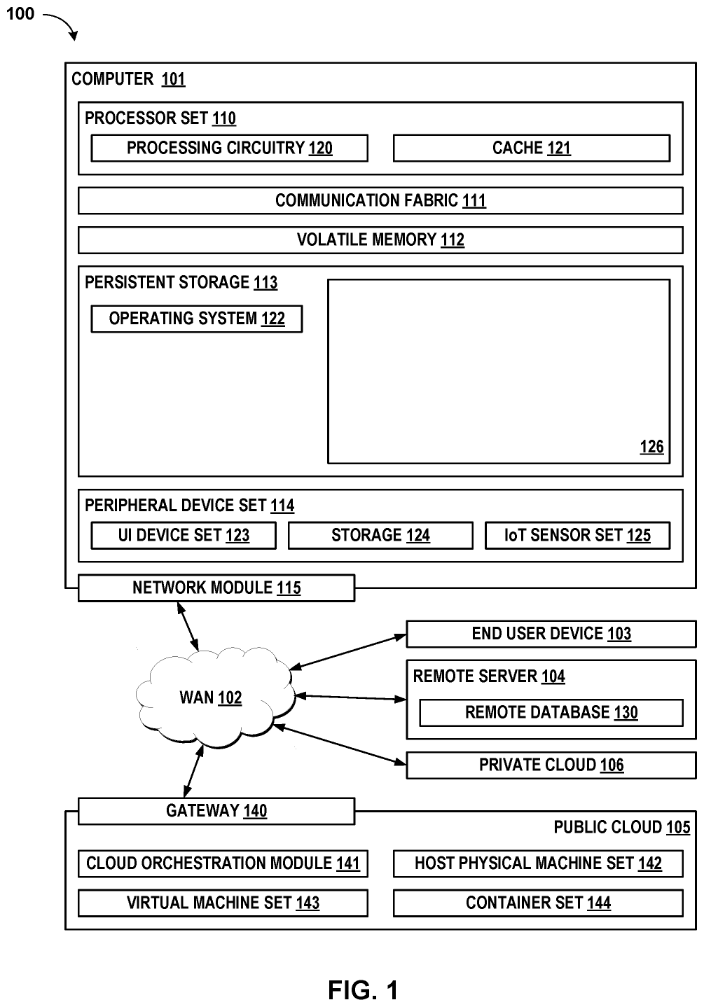

The following described exemplary embodiments provide a system, method and computer program that predicts diagnosis results based on unannotated cardiac auscultation text and audio data. Referring now to FIG. 1, Computing environment 100 contains an example of an environment for the execution of at least some of the computer code involved in performing the inventive methods, such as Cardiac Auscultation 126. In addition to Cardiac Auscultation 126, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and Cardiac Auscultation 126, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in Cardiac Auscultation 126 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in Cardiac Auscultation 126 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

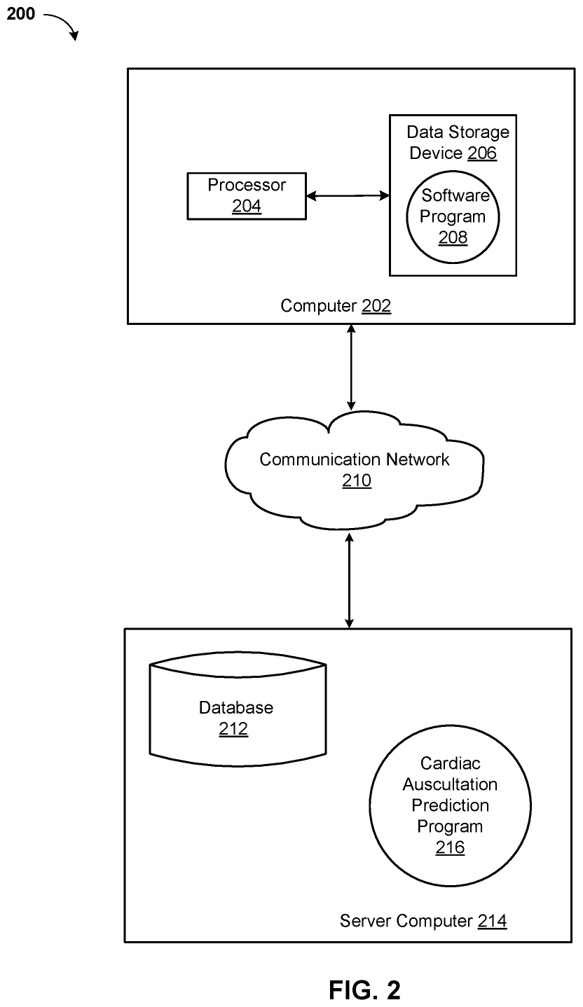

Referring now to FIG. 2, a functional block diagram of a networked computer environment illustrating a cardiac auscultation system 200 (hereinafter “system”) for predicting treatment options based on cardiac auscultation data. It should be appreciated that FIG. 2 provides only an illustration of one implementation and does not imply any limitations with regard to the environments in which different embodiments may be implemented. Many modifications to the depicted environments may be made based on design and implementation requirements.

The system 200 may include a computer 202 and a server computer 214. The computer 202 may communicate with the server computer 214 via a communication network 210 (hereinafter “network”). The computer 202 may include a processor 204 and a software program 208 that is stored on a data storage device 206 and is enabled to interface with a user and communicate with the server computer 214. The computer 202 may be, for example, a mobile device, a telephone, a personal digital assistant, a netbook, a laptop computer, a tablet computer, a desktop computer, or any type of computing devices capable of running a program, accessing a network, and accessing a database.

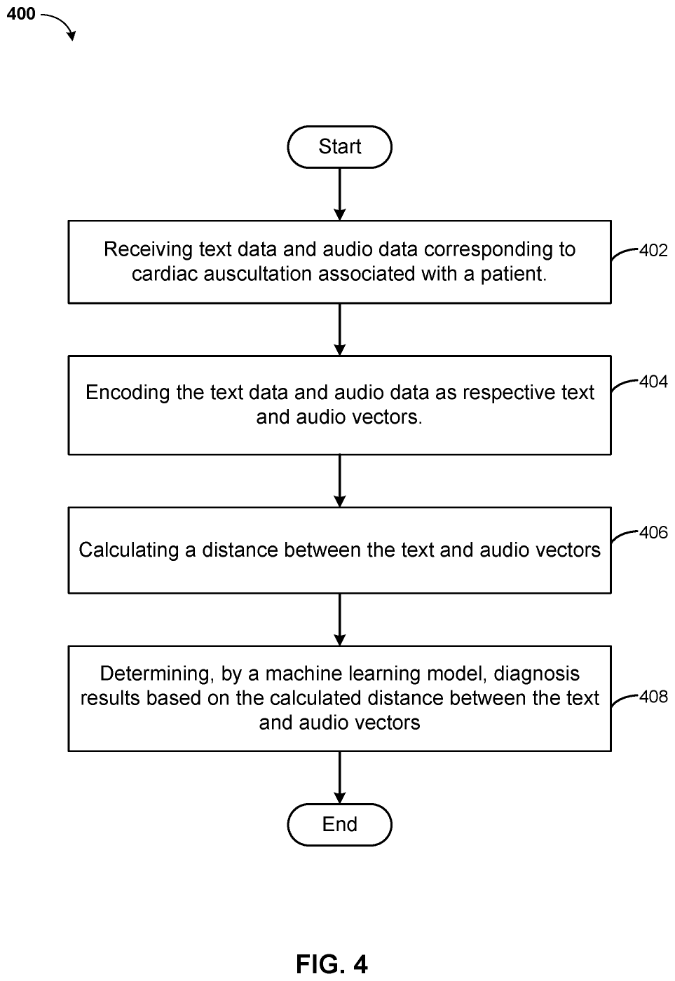

The server computer 214, which may be used for predicting treatment options based on cardiac auscultation data is enabled to run a Cardiac Auscultation Prediction Program 216 (hereinafter “program”) that may interact with a database 212. The Cardiac Auscultation Prediction Program is explained in more detail below with respect to FIG. 4. In one embodiment, the computer 202 may operate as an input device including a user interface while the program 216 may run primarily on server computer 214. In an alternative embodiment, the program 216 may run primarily on one or more computers 202 while the server computer 214 may be used for processing and storage of data used by the program 216. It should be noted that the program 216 may be a standalone program or may be integrated into a larger cardiac auscultation prediction program.

It should be noted, however, that processing for the program 216 may, in some instances be shared amongst the computers 202 and the server computers 214 in any ratio. In another embodiment, the program 216 may operate on more than one computer, server computer, or some combination of computers and server computers, for example, a plurality of computers 202 communicating across the network 210 with a single server computer 214. In another embodiment, for example, the program 216 may operate on a plurality of server computers 214 communicating across the network 210 with a plurality of client computers. Alternatively, the program may operate on a network server communicating across the network with a server and a plurality of client computers.

The network 210 may include wired connections, wireless connections, fiber optic connections, or some combination thereof. In general, the network 210 can be any combination of connections and protocols that will support communications between the computer 202 and the server computer 214. The network 210 may include various types of networks, such as, for example, a local area network (LAN), a wide area network (WAN) such as the Internet, a telecommunication network such as the Public Switched Telephone Network (PSTN), a wireless network, a public switched network, a satellite network, a cellular network (e.g., a fifth generation (5G) network, a long-term evolution (LTE) network, a third generation (3G) network, a code division multiple access (CDMA) network, etc.), a public land mobile network (PLMN), a private network, an ad hoc network, an intranet, a fiber optic-based network, or the like, and/or a combination of these or other types of networks.

The number and arrangement of devices and networks shown in FIG. 2 are provided as an example. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of system 200 may perform one or more functions described as being performed by another set of devices of system 200.

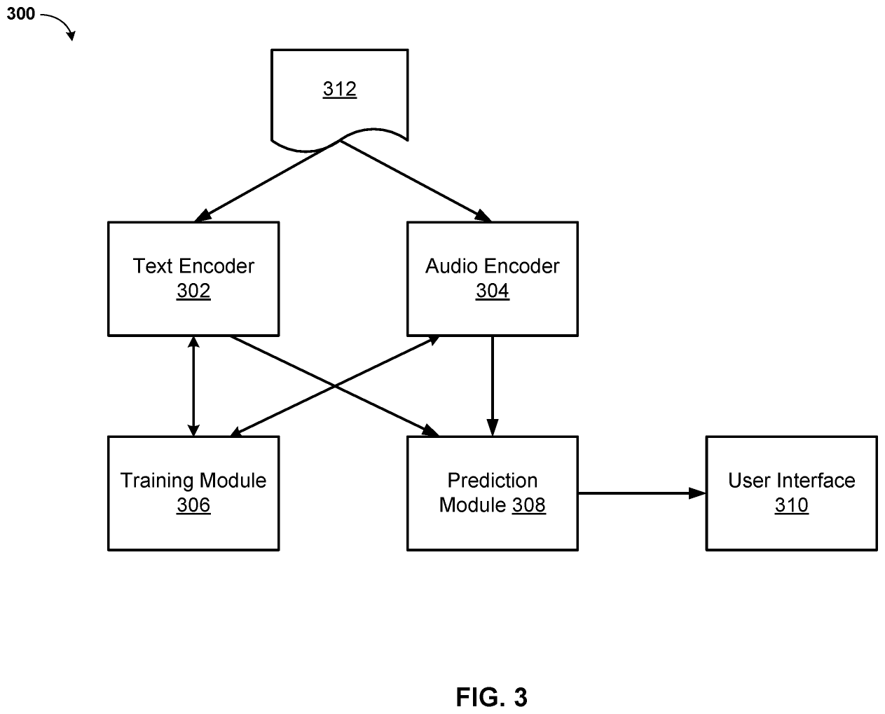

Referring now to FIG. 3, a cardiac auscultation system 300 is depicted according to one or more embodiments. The cardiac auscultation system 300 may use a text encoder and an audio encoder to select heart sound signals and description texts of auscultation results of the heart sound signal in corresponding medical records as training data and to determine positive samples and negative samples. Thus, the cardiac auscultation system 300 may include, among other things, a text encoder 302, an audio encoder 304, a training module 306, a prediction module 308, and a user interface 310.

The text encoder 302 may receive a text component of input data 312 that may correspond to descriptive text about cardiac auscultation in a patient's historical medical record. The text encoder 302 may determine a vector representation of the text component of the input data 312. Before the text component of the input data 312 is input into the model to start training, the text encoder 302 may extract key information, and then use natural language processing, such as a word-to-vector technique or a transformer model, to obtain the vector representation of the text component of the input data 312. The text encoder 302 may use, among other things, bidirectional encoder representations from transformers, generative pre-trained transformer, or residual neural network architectures to encode the text from the input data 312 as a vector Tₙ (e.g., T₁, T₂, . . . ) for each iteration of the text encoder 302.

The audio encoder 304 may receive an audio component of the input data 312 that may correspond to audio samples of heartbeat sound data associated with the descriptive text. Correspondingly, the audio component of the input data 312 may need to be preprocessed. Since the heart sound signal may be weak and may contain ambient noise, signal enhancement and filtering processing may be performed on the heart sound signal. The audio encoder 304 may then extract acoustic features, such as mel-frequency cepstral coefficients or logarithmic mel-frequency cepstral coefficients, from the audio component of the input data 312. The audio encoder may use a feature vector of the sound signal into the audio encoder to get audio vector Aₙ (e.g., A₁, A₂, . . . ) for each iteration of the audio encoder 304. The audio encoder 304 may utilize a variety of architectures, such as wave-to-vector transformer, sound convolutional neural networks, autoencoders, and the like.

The training module 306 may be used to train the cardiac auscultation system 300. The training module 306 may perform the training by minimizing the distance between the output vectors of positive samples input to the text encoder 302 and the audio encoder 304 and by maximizing the distance between the output vectors of negative samples input to the text encoder 302 and the audio encoder 304. Thus, the training module 306 may enable the trained text encoder 302 and the audio encoder 304 to communicate and introduce a supervision signal with each other, and then adjust the distribution of the data in the vector space. For example, if the text and audio components of the input data 312 are corresponding (i.e., a text entry of the medical record text of input data 312 corresponds to heart sound signal), this correspondence may be defined as positive samples. Otherwise, if the text and audio components do not correspond, such signals are negative samples, and a contrastive learning method may be used to train the model. Thus, the training module 306 may minimize the distance between positive samples and maximize the distance between negative samples.

The prediction module 308 may be used to predict the need for examination, treatment, or other intervention based on the outputs of the text encoder 302 and audio encoder 304. When the cardiac auscultation system 300 is successfully trained by the training module 306, the prediction module 308 may define a list of auscultation results, which can also be understood as some labels. When a portion of heart sound signal to be detected is received, the audio encoder 304 may receive a heart sound signal from the input data 312 as input to generate the audio vector Aₙ, which is input with all labels. The text vector Tₙ generated by the text encoder 302 may additionally be analyzed for similarity. If the determined similarity between the audio vector Aₙ and text vector Tₙ is less than a preset threshold, the prediction module 308 may identify that there is a related problem in this segment of the heart electrical signal. Further examination or treatment plans are recommended for the patient based on this result. After getting the trained model, the prediction module 308 can start to make predictions. The prediction module 308 may summarize a label list according to the cardiac auscultation audio and text data in the input data 312. The label list describes all possible symptoms after cardiac auscultation. Similarly, entity recognition may be performed before the tag list is converted into structured tags. The tags may be input to the text encoder 302 to get the respective feature vectors Tₙ (i.e., T₁, T₂. T₃ . . . ). When the patient's heart sound data in the input data 312 is input to the audio encoder 304, the audio encoder 304 may perform data preprocessing and acoustic feature extraction. The audio encoder 304 may obtain a feature vector Aₙ of the sound signal which may be passed to the prediction module 308. The prediction module 308 may compute the similarity between Aₙ and Tₙ for each iteration to obtain distance vector Dₙ (e.g., D₁, D₂, . . . ). The prediction module 308 may evaluate each distance vector Dₙ and compare them to a preset threshold value. If the distance is less than the preset threshold value, the two vectors are considered to be similar. For example, if D₂ and D₄ corresponding to particular heart sound results are less than the threshold value, the prediction module may determine that the result of cardiac auscultation may be “weak heart sounds and P2 split.”

The user interface 310 may display the results generated by the prediction module 308 to a user of the cardiac auscultation system 300. After analyzing the medical record data, the user interface may display a summarized diagnosis and treatment plan table to a user. The diagnosis and treatment plan table may be used to record future diagnostic results corresponding to different heart sound symptoms. The user interface 310 may, therefore, display the next treatment and examination plan recommended to the patient.

Thus, the cardiac auscultation system 300 may be used for providing diagnosis and treatment solutions for doctors. Additionally, because there is no need for fine-grained annotation data, the workload associated with annotating such data may be significantly reduced. Through the introduction of a supervision signal, the medical record text data and the acoustic data may be compared by a multimodal method to which may allow for adjustment of the acoustic vector space distribution.

Referring now to FIG. 4, an operational flowchart illustrating the steps of a method 400 carried out by a program that predicts treatment and intervention options based on cardiac auscultation text and audio data is depicted. The method 400 may be described with the aid of the exemplary embodiments of FIGS. 1-3.

At 402, the method 400 may include receiving text data and audio data corresponding to cardiac auscultation associated with a patient. The cardiac auscultation data comprises data corresponding to heart rate, heart rhythm, heart sounds, and heart abnormalities. In operation, the Cardiac Auscultation Prediction Program 216 (FIG. 2) on the server computer 214 (FIG. 2) may retrieve the input data 312 (FIG. 3) from the database 212 (FIG. 2) or may receive the input data 312 from the data storage device 206 (FIG. 2) on the computer 202 (FIG. 2) over the communication network 210 (FIG. 2). The input data 312 may contain an audio component and a text component.

At 404, the method 400 may include encoding the text data and audio data as respective text and audio vectors. The text data is encoded through a bidirectional encoder representation from transformers, a generative pre-trained transformer, and/or a residual neural network. The audio data is encoded through a wave-to-vector transformer, a sound convolutional neural network, and/or an autoencoder. In operation, the text encoder 302 (FIG. 3) and the audio encoder 304 (FIG. 3) may respectively encode the text and audio components of the input data 312 (FIG. 3) as respective text and audio vectors.

At 406, the method 400 may include calculating a distance between the text and audio vectors. A machine learning model may be used for determining diagnosis results based on the received text data and audio data. Training the machine learning model includes minimizing a first distance between positive text data and audio data samples and maximizing a second distance between negative text data and audio data samples. In operation, the prediction module 308 (FIG. 3) may calculate the distance between the text and audio vectors. The training module 306 (FIG. 3) may receive the text and audio vectors from the respective text encoder 302 (FIG. 3) and the audio encoder 304 (FIG. 3). The training module 306 may use the text and audio vector data to train the text encoder 302 and the audio encoder 304.

At 408, the method 400 may include determining diagnosis results based on the calculated distance between the text and audio vectors. The predicted diagnosis are transmitted results to a user for monitoring and treatment of the patient. In operation, the prediction module 308 (FIG. 3) may use the calculated distance between the text and audio vector to determine a diagnosis based on the input data 312 (FIG. 3). The prediction module 308 may display the diagnostic results to be displayed on the user interface 310 (FIG. 3) or may transmit the diagnostic results to the computer 202 (FIG. 2) over the communication network 210 (FIG. 2) and cause the software program 208 (FIG. 2) to display the diagnostic results.

It may be appreciated that FIG. 4 provides only an illustration of one implementation and does not imply any limitations with regard to how different embodiments may be implemented. Many modifications to the depicted environments may be made based on design and implementation requirements.

Some embodiments may relate to a system, a method, and/or a computer program product at any possible technical detail level of integration. The computer program product may include a computer-readable non-transitory storage medium (or media) having computer readable program instructions thereon for causing a processor to carry out operations.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program code/instructions for carrying out operations may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects or operations.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer readable media according to various embodiments. In this regard, each block in the flowchart or block diagrams may represent a module, segment, or portion of instructions, which comprises one or more executable instructions for implementing the specified logical function(s). The method, computer system, and computer program product may include additional blocks, fewer blocks, different blocks, or differently arranged blocks than those depicted in the Figures. In some alternative implementations, the functions noted in the blocks may occur out of the order noted in the Figures. For example, two blocks shown in succession may, in fact, be executed concurrently or substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

No element, act, or instruction used herein should be construed as critical or essential unless explicitly described as such. Also, as used herein, the articles “a” and “an” are intended to include one or more items and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The descriptions of the various aspects and embodiments have been presented for purposes of illustration but are not intended to be exhaustive or limited to the embodiments disclosed. Even though combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of predicting treatment options based on cardiac auscultation data, executable by a processor, comprising:
receiving text data and audio data corresponding to cardiac auscultation associated with a patient;
encoding the text data and the audio data as respective text vectors and audio vectors;
calculating a distance between the text vectors and the audio vectors; and
determining, by a machine learning model, diagnosis results based on the calculated distance between the text vectors and the audio vectors.

2. The method of claim 1, further comprising training the machine learning model for determining the diagnosis results based on the text data and the audio data.

3. The method of claim 2, wherein training the machine learning model comprises minimizing a first distance between positive samples of the text data and the audio data and maximizing a second distance between negative samples of the text data and the audio data.

4. The method of claim 1, wherein the text data and the audio data corresponding to the cardiac auscultation associated with the patient comprises data corresponding to heart rate, heart rhythm, heart sounds, and heart abnormalities.

5. The method of claim 1, wherein the text data is encoded through one or more from among a bidirectional encoder representation from transformers, a generative pre-trained transformer, and a residual neural network.

6. The method of claim 1, wherein the audio data is encoded through one or more from among a wave-to-vector transformer, a sound convolutional neural network, and an autoencoder.

7. The method of claim 1, further comprising transmitting the diagnosis results to a user for monitoring and treatment of the patient.

8. A computer system for predicting treatment options based on cardiac auscultation data, the computer system comprising:
one or more computer-readable storage media configured to store computer program code; and
one or more computer processors configured to access said computer program code stored on the one or more computer-readable storage media and operate as instructed by said computer program code, said computer program code including:
receiving code configured to cause the one or more computer processors to receiving text data and audio data corresponding to cardiac auscultation associated with a patient;
encoding code configured to cause the one or more computer processors to encode the text data and the audio data as respective text vectors and audio vectors;
calculating code configured to cause the one or more computer processors to calculate a distance between the text vectors and the audio vectors; and
determining code configured to cause the one or more computer processors to determine, by a machine learning model, diagnosis results based on the calculated distance between the text vectors and the audio vectors.

9. The computer system of claim 8, further comprising training code stored on the one or more computer-readable storage media, the training code configured to cause the one or more computer processors to train the machine learning model for determining the diagnosis results based on the text data and the audio data.

10. The computer system of claim 9, wherein training the machine learning model comprises minimizing a first distance between positive samples of the text data and the audio data and maximizing a second distance between negative samples of the text data and the audio data.

11. The computer system of claim 8, wherein the text data and the audio data corresponding to the cardiac auscultation associated with the patient comprises data corresponding to heart rate, heart rhythm, heart sounds, and heart abnormalities.

12. The computer system of claim 8, wherein the text data is encoded through one or more from among a bidirectional encoder representation from transformers, a generative pre-trained transformer, and a residual neural network.

13. The computer system of claim 8, wherein the audio data is encoded through one or more from among a wave-to-vector transformer, a sound convolutional neural network, and an autoencoder.

14. The computer system of claim 8, further comprising transmitting code stored on the one or more computer-readable storage media, the training code configured to cause the one or more computer processors to transmit the diagnosis results to a user for monitoring and treatment of the patient.

15. A computer program product for predicting treatment options based on cardiac auscultation data, comprising:
one or more computer-readable storage devices; and
program instructions stored on at least one of the one or more computer-readable storage devices, the program instructions configured to cause one or more computer processors to:
receive text data and audio data corresponding to cardiac auscultation associated with a patient;
encode the text data and the audio data as respective text vectors and audio vectors;
calculate a distance between the text vectors and the audio vectors; and
determine, by a machine learning model, diagnosis results based on the calculated distance between the text vectors and the audio vectors.

16. The computer program product of claim 15, wherein the program instructions stored on the one or more computer-readable storage devices are further configured to cause one or more computer processors train the machine learning model for determining the diagnosis results based on the text data and the audio data.

17. The computer program product of claim 16, wherein training the machine learning model comprises minimizing a first distance between positive samples of the text data and the audio data and maximizing a second distance between negative samples of the text data and the audio data.

18. The computer program product of claim 15, wherein the text data and the audio data corresponding to the cardiac auscultation associated with the patient comprises data corresponding to heart rate, heart rhythm, heart sounds, and heart abnormalities.

19. The computer program product of claim 15, wherein the text data is encoded through one or more from among a bidirectional encoder representation from transformers, a generative pre-trained transformer, and a residual neural network.

20. The computer program product of claim 15, wherein the audio data is encoded through one or more from among a wave-to-vector transformer, a sound convolutional neural network, and an autoencoder.
